SEQUENCE LISTING (4 sequences)

SEQ ID NO: 1 - DNA, 69 bp, Artificial
  CFTR-specific Snapback Probe
agggttgcgatggttctgttgtaggtactcgtgtcttttgacgagagcttgatgcgatctgtgagccga

SEQ ID NO: 2 - DNA, 114 bp, Artificial
  CFTR Template
gcagtgggctgtaaactccagcatagatgtggatagcttgatgcgatctgtgagccgagtctttaagttcattgacatgccaacagaaggtaaacctaccaagtcaaccaaacc

SEQ ID NO: 3 - DNA, 19 bp, Artificial
  PCR primer
gcagtgggctgtaaactcc

SEQ ID NO: 4 - DNA, 20 bp, Artificial
  PCR primer
ggtttggttgacttggtagg

(19) United States
(12) Patent Application Publication
(10) Pub. No.: US 2007/0020656 A1
(43) Pub. Date: Jan. 25, 2007

(54) Snapback oligonucleotide probe

(75) Inventor: Joseph A. Sorge, Del Mar, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/432,705

(22) Filed: May 10, 2006

Related U.S. Application Data
(60) Provisional application No. 60/679,934, filed on May 11, 2005.

(51) Int. Cl. C12Q 1/68; C12P 19/34; C07H 21/04

(52) U.S. Cl. 435/6; 536/24.3; 435/91.2

(57) ABSTRACT

The invention relates to compositions and methods for detection of specific nucleic acid sequences during an amplification reaction. The invention further relates to a kit format of said compositions for detection of nucleic acid sequences. The Snapback oligonucleotide probes of the invention comprise a target binding sequence, a hairpin forming sequence, and a reporter binding sequence. The action of a nucleic acid polymerase or a flap endonuclease cleaves off a “snapback segment” of the probe containing the hairpin forming sequence and reporter binding sequence, causing the probe to “snap back” and form a hairpin structure. The further action of a nucleic acid polymerase or a flap endonuclease cleaves off a label moiety from a reporter oligonucleotide hybridized to the reporter binding sequence, resulting in a detectable signal.

### RELATED APPLICATION

This application claims the benefit of U.S. Provisional Application No. 60/679,934 filed on May 11, 2005. The entire teachings of the above application are incorporated herein by reference.

### TECHNICAL FIELD

The invention relates to probes for the detection of nucleic acid sequences.

### BACKGROUND

Techniques for polynucleotide detection have found widespread use in basic research, diagnostics, and forensics. Polynucleotide detection can be accomplished by a number of methods. Most methods rely on the use of the polymerase chain reaction (PCR) to amplify the amount of target DNA.

The TaqMan™ assay is a homogenous assay for detecting polynucleotides (see U.S. Pat. No. 5,723,591). In this assay, two PCR primers flank a central probe oligonucleotide. The probe oligonucleotide contains a fluorophore and quencher. During the polymerization step of the PCR process, the 5′ nuclease activity of the polymerase cleaves the probe oligonucleotide, causing the fluorophore moiety to become physically separated from the quencher, which increases fluorescence emission. As more PCR product is created, the intensity of emission at the novel wavelength increases.

Molecular beacons are an alternative to TaqMan for the detection of polynucleotides (see U.S. Pat. Nos. 6,277,607; 6,150,097; and 6,037,130). Molecular beacons are oligonucleotide hairpins which undergo a conformational change upon binding to a perfectly matched template. The conformational change of the oligonucleotide increases the physical distance between a fluorophore moiety and a quencher moiety present on the oligonucleotide. This increase in physical distance causes the effect of the quencher to be diminished, thus increasing the signal derived from the fluorophore.

The adjacent probes method amplifies the target sequence by polymerase chain reaction in the presence of two nucleic acid probes that hybridize to adjacent regions of the target sequence, one of the probes being labeled with an acceptor fluorophore and the other probe labeled with a donor fluorophore of a fluorescence energy transfer pair. Upon hybridization of the two probes with the target sequence, the donor fluorophore interacts with the acceptor fluorophore to generate a detectable signal. The sample is then excited with light at a wavelength absorbed by the donor fluorophore and the fluorescent emission from the fluorescence energy transfer pair is detected for the determination of that target amount. U.S. Pat. No. 6,174,670B1 discloses such methods.

Sunrise primers utilize a hairpin structure similar to molecular beacons, but attached to a target binding sequence which serves as a primer. When the primer's complementary strand is synthesized, the hairpin structure is disrupted, thereby eliminating quenching. These primers detect amplified product and do not require the use of a polymerase with a 5′ exonuclease activity. Sunrise primers are described by Nazarenko et al. (Nucleic Acids Res. 25:2516-21 (1997) and in U.S. Pat. No. 5,866,336.

Scorpion probes combine a primer with an added hairpin structure, similar to Sunrise primers. However, the hairpin structure of Scorpion probes is not opened by synthesis of the complementary strand, but by hybridization of part of the hairpin structure with a portion of the target which is downstream from the portion which hybridizes to the primer.

DzyNA-PCR involves a primer containing the antisense sequence of a DNAzyme, an oligonucleotide capable of cleaving specific RNA phosphodiester bonds. The primer binds to a target sequence and drives an amplification reaction producing an amplicon which contains the active DNAzyme. The active DNAzyme then cleaves a generic reporter substrate in the reaction mixture. The reporter substrate contains a fluorophore-quencher pair, and cleavage of the substrate produces a fluorescence signal which increases with the amplification of the target sequence. Dzy-PCR is described in Todd et al., Clin. Chem. 46:625-30 (2000), and in U.S. Pat. No. 6,140,055.

### SUMMARY OF THE INVENTION

The invention is related to novel compositions and methods for nucleic acid detection.

The invention provides an oligonucleotide probe for detecting a target nucleic acid sequence. The probe comprises a target binding sequence, a hairpin forming sequence, and a reporter binding sequence. The portion of the probe comprising the hairpin forming sequence and the reporter binding sequence forms a cleavage structure when the probe is bound to a target nucleic acid. The cleavage structure is cleaved by the action of a nucleic acid polymerase or flap endonuclease, releasing the portion containing the hairpin forming sequence and the reporter binding sequence. After its release from the target nucleic acid, the hairpin forming sequence forms a hairpin structure. In some embodiments, hairpin structure can also be formed also prior to cleavage, but to a lesser extent than following cleavage. Extension of the stem of the hairpin structure by a nucleic acid polymerase results in release of a label moiety from the reporter, resulting in a detectable signal. The probes of the invention produce a detectable signal, such as an increase in fluorescence emission, upon amplification of their respective target sequence.

In some embodiments, the 3′ end of the reporter binding sequence is covalently linked to the 5′ end of said hairpin forming sequence, and the 3′ end of the hairpin forming sequence is covalently linked to the 5′ end of said target binding sequence. Optionally, a linker sequence can be interspersed between the 3′ end of the reporter binding sequence and the 5′ end of the hairpin forming sequence; preferably, such a linker, if present, is about 3 nucleotides.

In yet other embodiments, the oligonucleotide probe described above is hybridized to a reporter oligonucleotide that binds to the reporter binding sequence of the oligonucleotide probe. In certain embodiments the reporter oligonucleotide contains an interactive pair of labels, of which one label moiety is a quencher and the other label moiety is a fluorophore. The fluorophore is quenched when the reporter oligonucleotide is bound to the reporter binding region of said oligonucleotide probe, and is unquenched upon cleavage of the reporter oligonucleotide by the action of either a nucleic acid polymerase or a flap endonuclease.

The invention also provides methods of detecting a nucleic acid amplification product. One embodiment is a method comprising amplifying a target nucleic acid sequence in the presence of an oligonucleotide probe of the invention hybridized to a reporter oligonucleotide. The amplification step is carried out using a pair of primers specific for the target nucleic acid sequence and a nucleic acid polymerase having a 5′ to 3′ exonuclease activity. In certain embodiments a flap endonuclease is also present during amplification. In some embodiments, the nucleic acid polymerase lacks a 5′ to 3′ exonuclease activity and a flap endonuclease is present during amplification. The method also comprises the step of detecting the signal produced by the cleavage of a label moiety from the reporter oligonucleotide. Detection of the signal indicates detection of an amplification product derived from the target sequence.

The invention further provides a method of quantifying a target sequence in a sample. The method comprises the steps of (1) detecting a target nucleic acid sequence in a sample by the method described in the preceding paragraph and (2) comparing the signal to a standard curve to obtain the quantity of the target sequence in the sample. In some embodiments of this method, the step of detecting is performed while concurrently amplifying a nucleic acid comprising the target sequence from the sample using an amplification method such as polymerase chain reaction, e.g. by performing “real time” PCR.

The invention also provides a method of discriminating between a first and a second nucleic acid target sequence in a sample, when the target sequences differ by one or more nucleotides at a polymorphic site. The method comprises the steps of (1) detecting the first target nucleic acid sequence in a sample by the method described above using a first oligonucleotide probe having a target binding sequence which is fully complementary to the first target sequence; (2) detecting the second target nucleic acid sequence in a sample by the method described above using a second oligonucleotide probe having a target binding sequence which is fully complementary to the second target sequence; and (3) measuring whether said first probe or said second probe produces a detectable signal, thereby detecting the presence of either the first or second target sequence, or a mixture of both. In some embodiments, this method can be employed quantitatively to determine the amounts of different forms of a sequence (e.g., two or more alleles, wild type and mutation, etc.) are present in a sample. For example, the signals of the first probe and the second probe can be compared to a standard curve to obtain the quantities of the first and second target sequence in the sample.

A variation of the above method of determining whether a first or a second nucleic acid target sequence is present in a sample utilizes first and second probes which produce distinguishable signals upon amplification of their respective target sequences. For example, the probes can employ two different fluorophores, each with a characteristic emission wavelength. In this way, two or more variants at a polymorphic locus can be detected or quantified simultaneously.

The invention also provides kits for detecting a product of a nucleic acid amplification. The kits include the oligonucleotide probe of the invention, packaging therefor, and instructions for use of the probe. Optionally, the kits also include a reporter oligonucleotide of the invention. Further optional components of the kits are primers specific for one or more target nucleic acid sequences, one or more nucleic acid polymerases, a flap endonuclease, and a buffer or reaction mix containing nucleotide substrates.

Further features and advantages of the invention and further embodiments will become more fully apparent in the following description of the embodiments and drawings thereof, and from the claims.

### BRIEF DESCRIPTION OF DRAWINGS

FIGS. 1A and 1B illustrate detection of an amplified product of a PCR reaction using a Snapback oligonucleotide probe. FIG. 1A depicts the cleavage of the snapback segment (SB) from the probe, and FIG. 1B is an enlarged view depicting the formation of the hairpin structure and release of a label moiety (F) from the reporter oligonucleotide (RO).

FIG. 2 shows the nucleotide sequence of a Snapback oligonucleotide probe designed to detect CFTR. The sequence of the probe as depicted is SEQ ID NO: 1.

FIG. 3 shows amplification plots resulting from the detection of the indicated starting amounts of CFTR target sequence using the CFTR Snapback oligonucleotide probe of FIG. 2.

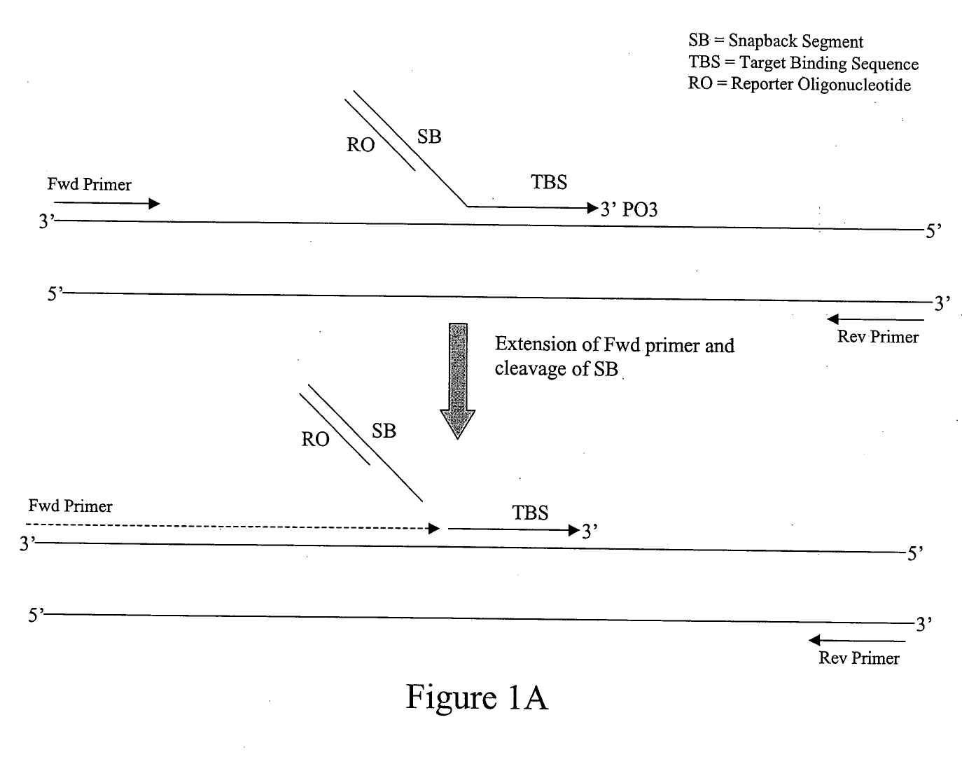

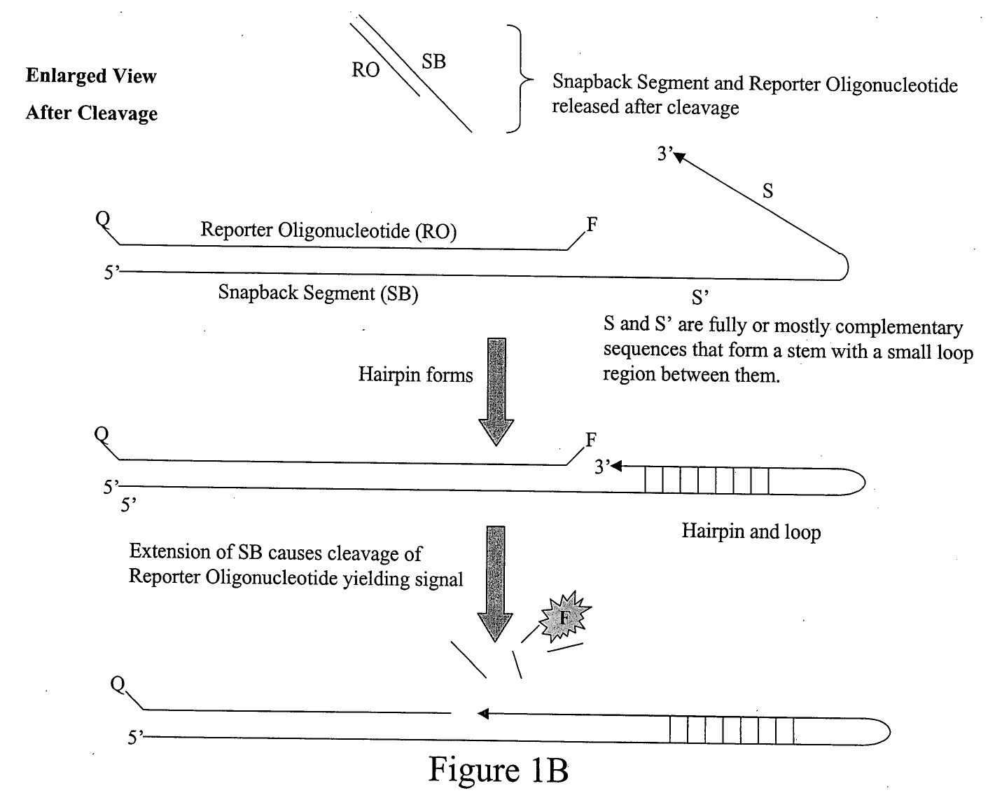

### DETAILED DESCRIPTION OF THE INVENTION

Definitions

As used herein, a “polynucleotide” refers to a covalently linked sequence of nucleotides (i.e., ribonucleotides for RNA and deoxyribonucleotides for DNA) in which the 3′ position of the pentose of one nucleotide is joined by a phosphodiester linkage to the 5′ position of the pentose of the next nucleotide. The term “polynucleotide” includes single- and double-stranded polynucleotides. The term “polynucleotide” as it is employed herein embraces chemically, enzymatically, or metabolically modified forms of polynucleotide. “Polynucleotide” also embraces a short polynucleotide, often referred to as an oligonucleotide (e.g., a primer or a probe). A polynucleotide has a “5′-terminus” and a “3′-terminus” because polynucleotide phosphodiester linkages occur between the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A “terminal nucleotide”, as used herein, is the nucleotide at the end position of the 3′- or 5′-terminus. As used herein, a polynucleotide sequence, even if internal to a larger polynucleotide (e.g., a sequence region within a polynucleotide), also can be said to have 5′- and 3′- ends.

Polynucleotides according to the invention may contain modified polynucleotides including locked nucleic acids (LNA), peptide nucleic acids (PNA), and the like. A PNA is a polyamide type of DNA analog, and the monomeric units for adenine, guanine, thymine and cytosine are available commercially (Applied Biosystems, Inc., Foster City, Calif.). Certain components of DNA, such as phosphorus, phosphorus oxides, or deoxyribose derivatives, are not present in PNAs. As disclosed by Nielsen et al., Science 254, 1497 (1991) and Egholm et al., Nature 365, 666 (1993), PNAs bind specifically and tightly to complementary DNA strands and are not degraded by nucleases. PNA/DNA duplexes bind under a wider range of stringency conditions than DNA/DNA duplexes, making it easier to perform multiplex hybridization. Smaller probes can be used than with DNA due to the strong binding. A single mismatch in a PNA/DNA 15-mer lowers the melting point (Tₘ) by 8-20 degrees C. vs. 4-16 degrees C. for the corresponding DNA/DNA 15-mer duplex. The absence of charged groups in PNA permits hybridization to be done at low ionic strengths. The synthesis and properties of LNAs are described in Koshkin et al., Tetrahedron, 54, 3607-3630 (1998) as well as in Wengel U.S. Pat. No. 6,794,499. LNA-containing oligonucleotides can be obtained commercially, for example from Proligo, LLP (Boulder, Colo.).

Furthermore, polynucleotides of the invention may comprise one or more modified bases selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 8-azaguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl)uracil, and 2,6-diaminopurine.

As used herein, the term “oligonucleotide” refers to a short polynucleotide, typically less than or equal to 150 nucleotides long (e.g., between 2 and 150, preferably between 10 and 100, more preferably between 15 and 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

As used herein, the term “complementary” refers to the concept of sequence complementarity between regions of two polynucleotide strands or between two regions of the same polynucleotide strand. It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” can refer to a first polynucleotide that is 100% or “fully” complementary to a second polynucleotide and thus forms a base pair at every nucleotide position. “Complementary” also can refer to a first polynucleotide that is not 100% complementary (e.g., 90%, 80%, 70% complementary or less) contains mismatched nucleotides at one or more nucleotide positions.

As used herein, the term “hybridization” or “binding” is used to describe the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved, the melting temperature (Tₘ) of the formed hybrid, the presence of other components (e.g., the presence or absence of polyethylene glycol), the molarity of the hybridizing strands, and the G:C content of the polynucleotide strands.

As used herein, when one polynucleotide is said to “hybridize” to another polynucleotide, it means that there is some complementarity between the two polynucleotides or that the two polynucleotides form a hybrid under high stringency conditions. When one polynucleotide is said to not hybridize to another polynucleotide, it means that there is essentially no sequence complementarity between the two polynucleotides or that no hybrid forms between the two polynucleotides at a high stringency condition. In one embodiment, two complementary polynucleotides are capable of hybridizing to each other under high stringency hybridization conditions. Hybridization under stringent conditions is typically established by performing membrane hybridization (e.g., Northern hybridization) under high stringency hybridization conditions, defined as incubation with a radiolabeled probe in 5X SSC, 5X Denhardt's solution, 1% SDS at 65° C. Stringent washes for membrane hybridization are performed as follows: the membrane is washed at room temperature in 2X SSC/0.1% SDS and at 65° C. in 0.2X SSC/0.1% SDS, 10 minutes per wash, and exposed to film.

As used herein, a “primer” refers to a type of oligonucleotide having or containing the length limits of an “oligonucleotide” as defined above, and having or containing a sequence complementary to a target polynucleotide, which hybridizes to the target polynucleotide through base pairing so to initiate an elongation (extension) reaction to incorporate a nucleotide into the oligonucleotide primer. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present invention are generally between about 10 and 1000 nucleotides in length, preferably between about 14 and 50 nucleotides in length, and most preferably between about 17 and 45 nucleotides in length. An “amplification primer” is a primer for amplification of a target sequence by primer extension. As no special sequences or structures are required to drive the amplification reaction, amplification primers for PCR may consist only of target binding sequences. A “primer region” is a region on an “oligonucleotide probe” or a “bridging oligonucleotide probe” which hybridizes to the target nucleic acid through base pairing so to initiate an elongation reaction to incorporate a nucleotide into the oligonucleotide primer.

As used herein, a polynucleotide “isolated” from a sample is a naturally occurring polynucleotide sequence within that sample which has been removed from its normal cellular environment. Thus, an “isolated” polynucleotide may be in a cell-free solution or placed in a different cellular environment.

As used herein, the term “amount” refers to an amount of a target polynucleotide in a sample, e.g., measured in μg, μmol or copy number. The abundance of a polynucleotide in the present invention is measured by the fluorescence intensity emitted by such polynucleotide, and compared with the fluorescence intensity emitted by a reference polynucleotide, i.e., a polynucleotide with a known amount.

As used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. A “probe which shares no homology with another polynucleotide” refers to a probe whose homology to the polynucleotide, as measured by BLASTN version 2.0 using the default parameters, is no more than 55%, e.g., less than 50%, or less than 45%, or less than 40%, or less than 35%, in a contiguous region of 20 nucleotides or more.

A “hairpin structure”, as used herein, comprises two self-complementary sequences that may form a double-stranded “stem” region, optionally separated at one end by a loop sequence. The two regions of the reporter oligonucleotide which form the double-stranded stem region are substantially complementary to each other, resulting in self-hybridization. However, the stem can include one or more mismatches, insertions or deletions. The “hairpin structure”, as used herein, can additionally comprise single-stranded region(s) that extend from the double-stranded stem segment.

As used herein, “Tₘ” and “melting temperature” are interchangeable terms which are the temperature at which 50% of a population of double-stranded polynucleotide molecules becomes dissociated into single strands. Equations for estimating the Tₘ of polynucleotides are well known in the art. For example, the Tₘ may be estimated by the following equation: Tₘ=69.3+0.41 X (G+C) %−650/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate which can be used to predict an appropriate temperature for a given hybridization or dissociation step; the optimum temperature is commonly determined empirically.

A “nucleotide analog”, as used herein, refers to a nucleotide in which the pentose sugar and/or one or more of the phosphate esters are replaced with their respective analogs. Exemplary pentose sugar analogs are those previously described in conjunction with nucleoside analogs. Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. Also included within the definition of “nucleotide analog” are nucleobase monomers which can be polymerized into polynucleotide analogs in which the DNA/RNA phosphate ester and/or sugar phosphate ester backbone is replaced with a different type of linkage. A nucleotide analog can also be an LNA or a PNA (see above).

As used herein, the term “sample” refers to a biological material which is isolated from its natural environment and contains a polynucleotide. A “sample” according to the invention may consist of purified or isolated polynucleotide, or it may comprise a biological sample such as a tissue sample, a biological fluid sample, or a cell sample comprising a polynucleotide. A biological fluid can be, for example, blood, plasma, sputum, urine, cerebrospinal fluid, lavages, and leukophoresis samples. A sample of the present invention may be a plant, animal, bacterial or viral material containing a target polynucleotide. Useful samples of the present invention may be obtained from different sources, including, for example, but not limited to, from different individuals, different developmental stages of the same or different individuals, different diseased individuals, normal individuals, different disease stages of the same or different individuals, individuals subjected to different disease treatments, individuals subjected to different environmental factors, individuals with predisposition to a pathology, individuals with exposure to an infectious disease (e.g., HIV). Useful samples may also be obtained from in vitro cultured tissues, cells, or other polynucleotide containing sources. The cultured samples may be taken from sources including, but are not limited to, cultures (e.g., tissue or cells) cultured in different media and conditions (e.g., pH, pressure, or temperature), cultures (e.g., tissue or cells) cultured for different period of length, cultures (e.g., tissue or cells) treated with different factors or reagents (e.g., a drug candidate, or a modulator), or cultures of different types of tissue or cells.

As used herein, “nucleic acid polymerase” refers to an enzyme that catalyzes the polymerization of nucleotides. Generally, the enzyme will initiate synthesis at the 3′-end of the primer annealed to a nucleic acid template sequence, and will proceed toward the 5′ end of the template strand. “DNA polymerase” catalyzes the polymerization of deoxyribonucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19: 4193), 9°Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzyme can be determined by means well known in the art. One unit of DNA polymerase activity, according to the subject invention, is defined as the amount of enzyme which catalyzes the incorporation of 10 nmoles of total dNTPs into polymeric form in 30 minutes at optimal temperature (e.g., 72° C. for Pfu DNA polymerase).

“Primer extension reaction” or “synthesizing a primer extension” means a reaction between a target-primer hybrid and a nucleotide which results in the addition of the nucleotide to a 3′-end of the primer such that the incorporated nucleotide is complementary to the corresponding nucleotide of the target polynucleotide. Primer extension reagents typically include (i) a polymerase enzyme, (ii) a buffer, and (iii) one or more extendible nucleotides.

As used herein, “polymerase chain reaction” or “PCR” refers to an in vitro method for amplifying a specific polynucleotide template sequence. The PCR reaction involves a repetitive series of temperature cycles and is typically performed in a volume of 25-100 μl. The reaction mix comprises dNTPs (each of the four deoxyribonucleotides dATP, dCTP, dGTP, and dTTP), primers, buffers, DNA polymerase, and polynucleotide template. One PCR reaction may consist of 5 to 100 “cycles” of denaturation and synthesis of a polynucleotide molecule. The PCR process is described in U.S. Pat. Nos. 4,683,195 and 4,683,202, the disclosures of which are incorporated herein by reference.

As used herein a “nuclease” or a “cleavage agent” refers to an enzyme that is specific for, that is, cleaves a “cleavage structure” according to the invention and is not specific for, that is, does not substantially cleave either a probe or a primer that is not hybridized to a target nucleic acid, or a target nucleic acid that is not hybridized to a probe or a primer. The term “nuclease” includes an enzyme that possesses 5′ endonucleolytic activity for example a DNA polymerase, e.g. DNA polymerase I from E. coli, and DNA polymerase from Thermus aquaticus (Taq), Thermus thermophilus (Tth), Pyrococcus furiosus (Pfu) and Thermus flavus (Tfl). The term “nuclease” also embodies FEN nucleases.

As used herein a “flap” refers to a region of single stranded DNA that extends from a double stranded nucleic acid molecule. The length of a flap according to the invention is preferably in the range from about 1 to about 500 nucleotides, more preferably from about 5 to about 25 nucleotides, and most preferably from about 10 to about 20 nucleotides.

As used herein, a “cleavage structure” refers to a polynucleotide structure comprising at least a duplex nucleic acid having a single stranded region comprising a flap, a loop, a single-stranded bubble, a D-loop, a nick or a gap. A cleavage structure according to the invention thus includes a polynucleotide structure comprising a flap strand of a branched DNA wherein a 5′ single-stranded polynucleotide flap extends from a position near its junction to the double stranded portion of the structure, and preferably the flap is labeled with a detectable label. A flap of a cleavage structure according to the invention is preferably cleaved at a position located either one nucleotide proximal to and/or one nucleotide distal from the elbow of the flap strand. In some embodiments, a flap of a cleavage structure does not hybridize to a target nucleic acid sequence.

A cleavage structure according to one embodiment of the invention preferably comprises a target nucleic acid sequence, and also may include an oligonucleotide probe according to the invention, hybridized with the target nucleic acid sequence via a region or regions that are complementary to the target nucleic acid, and a 5′-flap extending from the hybridizing oligonucleotide probe.

FEN-1 is an approximately 40 kDa, divalent metal ion-dependent exo- and endonuclease that specifically recognizes the backbone of a 5′ single-stranded flap strand and tracks down this arm to the cleavage site, which is located at the junction wherein the two strands of duplex DNA adjoin the single-stranded arm. Both the endo- and exonucleolytic activities show little sensitivity to the base at the most 5′ position at the flap or nick. Both FEN-1 endo- and exonucleolytic substrate binding and cutting are stimulated by an upstream oligonucleotide (flap adjacent strand or primer). This is also the case for E. coli pol I. The endonuclease activity of the enzyme is independent of the 5′ flap length, cleaving a 5′ flap as small as one nucleotide. The endonuclease and exonuclease activities are insensitive to the chemical nature of the substrate, cleaving both DNA and RNA.

fen-I genes encoding FEN-1 enzymes useful in the invention include murinefen-1, human fen-1, rat fen-1, Xenopus laevis fen-1, and fen-1 genes derived from four archaebacteria Archaeglobus fulgidus, Methanococcus jannaschii, Pyrococcus furiosus and Pyrococcus horikoshii. CDNA clones encoding FEN-1 enzymes have been isolated from human (GenBank Accession Nos.: NM.sub.—004111 and L37374), mouse (GenBank Accession No.: L26320), rat (GenBank Accession No.: AA819793), Xenopus laevis (GenBank Accession Nos.: U68141 and U64563), and P. furiosus (GenBank Accession No.: AF013497). The complete nucleotide sequence for P. horikoshii flap endonuclease has also been determined (GenBank Accession No.: AB005215). The FEN-1 family also includes the Saccharomyces cerevisiae RAD27 gene (GenBank Accession No.: Z28113 Y13137) and the Saccharomyces pombe RAD2 gene (GenBank Accession No.: X77041). The archaeal genome of Methanobacterium thermautotrophiculum has also been sequenced. Although the sequence similarity between FEN-1 and prokaryotic and viral 5′ {character pullout} 3′ exonucleases is low, FEN-1s within the eukaryotic kingdom are highly conserved at the amino acid level, with the human and S. cerevisiae proteins being 60% identical and 78% similar. The three archaebacterial FEN-1 proteins are also highly homologous to the eukaryotic FEN-1 enzymes (reviewed in Matsui et al., 1999., J. Biol. Chem., 274:18297, Hosfield et al., 1998b, J. Biol. Chem., 273:27154 and Lieber, 1997, BioEssays, 19:233).

A FEN nuclease according to the invention is preferably thermostable. Thermostable FEN nucleases have been isolated and characterized from a variety of thermostable organisms including four archeaebacteria. The cDNA sequence (GenBank Accession No.: AF013497) and the amino acid sequence (Hosfield et al., 1998a, supra and Hosfield et al., 1998b) for P. furiosus flap endonuclease have been determined. The complete nucleotide sequence (GenBank Accession No.: AB005215) and the amino acid sequence (Matsui et al., supra) for P. horikoshii flap endonuclease have also been determined. The amino acid sequence for M. jannaschii (Hosfield et al., 1998b and Matsui et al., 1999 supra) and A. fulgidus (Hosfield et al., 1998b) flap endonuclease have also been determined.

As used herein, “5′ to 3′ exonuclease activity” or “5′→3′ exonuclease activity” refers to that activity of a template-specific nucleic acid polymerase e.g. a 5′→3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow (Klenow et al., 1970, Proc. Natl. Acad. Sci., USA, 65:168) fragment does not, (Klenow et al., 1971, Eur. J. Biochem., 22:371)), or polynucleotides are removed from the 5′ end by an endonucleolytic activity that may be inherently present in a 5′ to 3′ exonuclease activity.

As used herein, the phrase “substantially lacks 5′ to 3′ exonuclease activity” or “substantially lacks 5′→3′ exonuclease activity” means having less than 10%, 5%, 1%, 0.5%, or 0.1% of the activity of a wild type enzyme. The phrase “lacking 5′ to 3′ exonuclease activity” or “lacking 5′→3′ exonuclease activity” means having undetectable 5′ to 3′ exonuclease activity or having less than about 1%, 0.5%, or 0.1% of the 5′ to 3′ exonuclease activity of a wild type enzyme. 5′ to 3′ exonuclease activity may be measured by an exonuclease assay which includes the steps of cleaving a nicked substrate in the presence of an appropriate buffer, for example 10 mM Tris-HCl (pH 8.0), 10 mM MgCl₂ and 50 μg/ml bovine serum albumin) for 30 minutes at 60° C., terminating the cleavage reaction by the addition of 95% formamide containing 10 mM EDTA and 1 mg/ml bromophenol blue, and detecting nicked or un-nicked product.

Nucleic acid polymerases useful in certain embodiments of the invention substantially lack 3′ to 5′ exonuclease activity and include but are not limited to exo- Pfu DNA polymerase (a mutant form of Pfu DNA polymerase that substantially lacks 3′ to 5′ exonuclease activity, Cline et al., 1996, Nucleic Acids Research, 24: 3546; U.S. Pat. No. 5,556,772; commercially available from Stratagene, La Jolla, Calif. Catalogue # 600163), exo- Tma DNA polymerase (a mutant form of Tma DNA polymerase that substantially lacks 3′ to 5′ exonuclease activity), exo- Tli DNA polymerase (a mutant form of Tli DNA polymerase that substantially lacks 3′ to 5′ exonuclease activity New England Biolabs, (Cat #257)), exo- E. coli DNA polymerase (a mutant form of E. coli DNA polymerase that substantially lacks 3′ to 5′ exonuclease activity) exo-Klenow fragment of E. coli DNA polymerase I (Stratagene, Cat #600069), exo- T7 DNA polymerase (a mutant form of T7 DNA polymerase that substantially lacks 3′ to 5′ exonuclease activity), exo- KOD DNA polymerase (a mutant form of KOD DNA polymerase that substantially lacks 3′ to 5′ exonuclease activity), exo- JDF-3 DNA polymerase (a mutant form of JDF-3 DNA polymerase that substantially lacks 3′ to 5′ exonuclease activity), exo- PGB-D DNA polymerase (a mutant form of PGB-D DNA polymerase that substantially lacks 3′ to 5′ exonuclease activity) New England Biolabs, Cat. #259, Tth DNA polymerase, Taq DNA polymerase (e.g., Cat. Nos. 600131, 600132, 600139, Stratagene); UlTma (N-truncated) Thermatoga martima DNA polymerase; Klenow fragment of DNA polymerase I, 9°Nm DNA polymerase (discontinued product from New England Biolabs, Beverly, Mass.), “3′-5′ exo reduced” mutant (Southworth et al., 1996, Proc. Natl. Acad. Sci 93:5281) and Sequenase (USB, Cleveland, Ohio). The polymerase activity of any of the above enzyme can be defined by means well known in the art. One unit of DNA polymerase activity, according to the subject invention, is defined as the amount of enzyme which catalyzes the incorporation of 10 nmoles of total dNTPs into polymeric form in 30 minutes at optimal temperature.

As used herein, “amplification” refers to any in vitro method for increasing the number of copies of a nucleic acid template sequence with the use of a polymerase. Nucleic acid amplification results in the incorporation of nucleotides into a nucleic acid (e.g., DNA) molecule or primer thereby forming a new nucleic acid molecule complementary to the nucleic acid template. The formed nucleic acid molecule and its template can be used as templates to synthesize additional nucleic acid molecules. As used herein, one amplification reaction may consist of many rounds of nucleic acid synthesis. Amplification reactions include, for example, polymerase chain reactions (PCR; Mullis and Faloona, 1987, Methods Enzymol. 155:335). One PCR reaction may consist of 5 to 100 “cycles” of denaturation and synthesis of a nucleic acid molecule. PCR amplifications with an exo- DNA polymerase inherently will result in generating mutated amplified product.

As used herein, “polymerase chain reaction” or “PCR” refers to an in vitro method for amplifying a specific nucleic acid template sequence. The PCR reaction involves a repetitive series of temperature cycles and is typically performed in a volume of 25-100 μl. The reaction mix comprises dNTPs (each of the four deoxyribonucleotides dATP, dCTP, dGTP, and dTTP), primers, buffers, DNA polymerase, and nucleic acid template. The PCR reaction comprises providing a set of oligonucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of oligonucleotide primers” or “a set of PCR primers” can comprise two, three, four or more primers. In one embodiment, an exo- Pfu DNA polymerase is used to amplify a nucleic acid template in a PCR reaction.

As used herein, the term “PCR primer” refers to a single stranded DNA or RNA molecule that can hybridize to a nucleic acid template and prime enzymatic synthesis of a second nucleic acid strand. A PCR primer useful according to the invention is between 10 to 100 nucleotides in length, preferably 17-50 nucleotides in length and more preferably 17-45 nucleotides in length. In some embodiments, primers of the invention comprise a tag or label that produces a secondary signal which is useful for detection. A tag can be, for example, an additional nucleic acid sequence that can be bound by a secondary sequence to produce a secondary signal.

As used herein, a “PCR reaction buffer” or “reaction buffer” refers to a single buffer composition which allows PCR amplification of a nucleic acid template by a nucleic acid polymerase. The buffer may contain any known chemicals used in a buffer for PCR reactions. Preferably, the buffer contains a buffering composition selected from Tris or Tricine. Preferably, the buffering composition has a pH range of from 7.5 to 9.5. Preferably, the PCR reaction buffer contains Mg²⁺ (e.g., MgCl₂ or MgSO₄) in the range of 1-10 mM. The buffer according to the invention may also contain K⁺ (e.g., KCl) in the range of from 0 to 20 mM. In some embodiments, the buffer contains components which enhance PCR yield (e.g., (NH₄)₂SO₄ in the range of from 0 to 20 mM). In other embodiments, the buffer contains one or more non-ionic detergents (e.g., Trition X-100, Tween 20, or NP40, in the range of from 0 to 1%). The buffer may also contain BSA (bovine serum albumin) in the range of from 1-100 μg/ml. In a preferred embodiment of the invention, the PCR reaction buffer contains 10 mM KCl, 10 mM (NH₄)₂SO₄, 20 mM Tris-Cl (pH 8.8), 2 mM MgSO₄, 0.1% Triton X-100, 100 μg/ml BSA. In another preferred embodiment, the buffer contains 10 mM KCl, 10 mM (NH₄)₂SO₄, 20 mM Tris-Cl (pH 9.2), 2 mM MgSO₄, 0.1% Triton X-100, 100 μg/ml BSA.

As used herein, the term “equivalent amount(s)” refers to components (e.g., dATP, dTTP, dGTP, and dCTP) in the PCR buffer having an equal molar concentration.

As used herein, an “amplified product” refers to the double stranded nucleic acid population at the end of a PCR amplification reaction. The amplified product contains the original nucleic acid template and nucleic acid synthesized by DNA polymerase using the nucleic acid template during the PCR reaction. The amplified product, according to the invention, contains mutations to the original nucleic acid template sequence due to the use of error-prone DNA polymerases in the PCR reaction, e.g., Mutazyme and Taq DNA polymerases.

As used herein, the term “repeating one or more additional subsequent PCR amplification reactions” refers to the subsequent performance of one or more additional PCR amplification reactions comprising incubating a nucleic acid template, at least two PCR primers, an error-prone DNA polymerase under conditions which permit amplification of the nucleic acid template. A subsequent PCR reaction comprises said incubating step using the PCR amplified product of a preceding PCR amplification as template. The amplified product of a preceding PCR amplification reaction may be purified before being used as template for a subsequent PCR reaction by means known in the art, e.g., phenol extraction/ethanol precipitation or column purification. The template for a subsequent PCR amplification reaction may be a portion of or the total amplified product of a preceding PCR amplification. For each subsequent PCR amplification, fresh reagents (e.g., reaction buffer, dNTP, DNA polymerase, primers) are added to the reaction mixture. If a portion of the amplified product of a preceding PCR amplification is used, the volume of a subsequent PCR reaction may be the same as the preceding PCR reaction. If the total amplified product of a preceding PCR reaction is used as template, a subsequent PCR reaction will have larger volume than the preceding PCR reaction.

As used herein, “nucleic acid template” or “target nucleic acid template” refers to a nucleic acid containing an amplified region. The “amplified region,” as used herein, is a region of a nucleic acid that is to be either synthesized or amplified by polymerase chain reaction (PCR). For example, an amplified region of a nucleic acid template resides between two sequences to which two PCR primers are complementary.

### DESCRIPTION

The inventors have discovered that oligonucleotide probes for the detection of a target nucleic acid sequence during an amplification reaction, for example during real time or quantitative PCR analysis, can be formed by joining three sequences: a target binding sequence, a hairpin forming sequence, and a reporter binding sequence. When the target binding sequence hybridizes to a target nucleic acid, a 5′-flap is formed; the flap contains the “snapback segment” of the probe, which includes the reporter binding sequence and the hairpin forming sequence. The 5′-flap is cleaved during the elongation period of an amplification reaction cycle by the action of either a flap endonuclease present in the reaction mixture or a nucleic acid polymerase having 5′ to 3′ exonuclease activity, during extension of the forward primer. Cleavage of the 5′-flap promotes the formation of a hairpin structure from the hairpin forming sequence. The stem of the hairpin is also extended during the extension period of the amplification reaction cycle. A reporter oligonucleotide is hybridized to the reporter binding sequence of the snapback segment. The reporter contains an interactive pair of labels, for example a fluorophore/quencher pair; each label or quencher is a “label moiety.” One label moiety of the pair is attached to the reporter at or near its 3′ end. The other label moiety is attached at the 5′ end of the reporter, such that the 5′ label moiety is cleaved off by the 5′ to 3′ exonuclease activity of a nucleic acid polymerase or by the action of a flap endonuclease. The probes are termed “Snapback Probes.”

Preparation of Primers and Probes

Oligonucleotide probes and primers can be synthesized by any method described below and other methods known in the art. Probes and primers are typically prepared by biological or chemical synthesis, although they can also be prepared by biological purification or degradation, e.g., endonuclease digestion. For short sequences such as probes and primers used in the present invention, chemical synthesis is frequently more economical as compared to biological synthesis. For longer sequences standard replication methods employed in molecular biology can be used such as the use of M13 for single stranded DNA as described by Messing, 1983, Methods Enzymol. 101: 20-78. Chemical methods of polynucleotide or oligonucleotide synthesis include phosphotriester and phosphodiester methods (Narang, et al., Meth. Enzymol. (1979) 68:90) and synthesis on a support (Beaucage, et al., Tetrahedron Letters. (1981) 22:1859-1862) as well as phosphoramidate technique, Caruthers, M. H., et al., Methods in Enzymology (1988)154:287-314 (1988), and others described in “Synthesis and Applications of DNA and RNA,” S. A. Narang, editor, Academic Press, New York, 1987, and the references contained therein. Probes of the invention can be formed from a single strand which assumes a secondary structure, typically a hairpin or similar structure, or can be formed from two or more single strands which associate, for example by hybridization of complementary bases, to form a hairpin or similar structure. Labels can be attached at any position on the reporter oligonucleotide, provided that a detectable signal is produced when a label moiety is cleaved off from the reporter oligonucleotide as a result of the target binding sequence of the probe hybridizing to the target sequence.

According to the present invention, the oligonucleotide probe can comprise natural, non-natural nucleotides and analogs. The probe may be a nucleic acid analog or chimera comprising nucleic acid and nucleic acid analog monomer units, such as 2-aminoethylglycine. For example, part or all of the probe may be PNA or a PNA/DNA chimera. Oligonucleotides with minor groove binders (MGBs), locked nucleic acids (LNA) and other modified nucleotides can be used. These oligonucleotides using synthetic nucleotides can have the advantage that the length can be shortened while maintaining a high melting temperature.

Probe Design

The probe of the present invention is ideally less than about 150 or less than about 130 nucleotides in length, typically less than about 100 nucleotides, for example less than about 90, 80, 70, or 60 nucleotides in length. Preferably, the probe of the invention is between about 10 and about 90 nucleotides in length, or between about 20 and about 80, more preferably between about 30 and about 70.

The target binding sequence of the probe is designed such that hybridization to target DNA occurs at the annealing/extension temperature of PCR. Therefore, the target binding sequence of the probe shares homology with the target DNA, whereas the reporter binding sequence and hairpin forming sequence typically do not share homology with the target nucleic acid sequence, although they may share limited homology to the target sequence over short stretches of up to 3, 4, or 5 nucleotides. The region of the target nucleic acid which is complementary to the target binding sequence is ideally located within 200 nucleotides downstream of (i.e., to the 3′ of) a primer binding site, typically within 150, 125, or 100 nucleotides of a primer binding site, when used in conjunction with PCR.

The target binding sequence of the probe is at least 5 nucleotides in length. In preferred embodiments, the target binding sequence is about 15 to about 60 nucleotides in length. In more preferred embodiments, the target binding sequence is about 15 to about 30 nucleotides in length. In certain embodiments, the target binding sequence is about 10 to about 15 nucleotides in length and optionally includes at least one modified nucleotide that increases the binding affinity of the probe for the target. Introduction of such modified nucleotides can be used to increase the affinity for the target sequence, to reduce the length of the target binding sequence, or to reduce the number of mismatches required for a desired affinity for the target sequence.

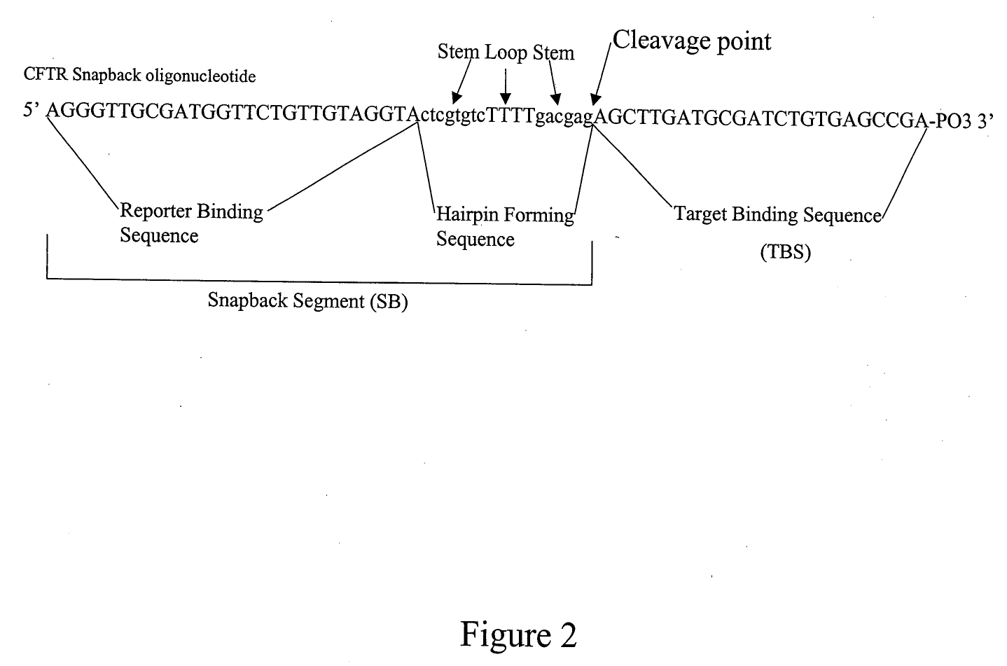

Hairpin structures are subject to denaturation at appropriate conditions, including high temperatures, reduced ionic concentrations, and/or the presence of disruptive chemical agents such as formamide or DMSO. Following cleavage at the cleavage point between the target binding sequence and the snapback segment (see, e.g., FIG. 2), the probes of the present invention form hairpin structure at the annealing/extension temperature, which is typically in the range of 55-65° C. Therefore, probes with a hairpin structure Tₘ higher than the annealing/extension temperature are preferred. For example, the hairpin structure of the probe can have a Tₘ≧55° C., ≧60° C., ≧62° C., or ≧65° C. However, Tₘ generally should not be more than about 15° C. higher than the annealing/extension temperature. In some embodiments the probe has a hairpin structure with Tₘ in the range from about the temperature of annealing of the target binding sequence to the target nucleic acid sequence (which can be chosen as the extension temperature as well) to about 5 to 15° C. above that temperature. The stability and melting temperature of hairpin structures can be estimated, for example, using programs such as mfold (Zuker (1989) Science, 244, 48-52) or Oligo 5.0 (Rychlik & Rhoads (1989) Nucleic Acids Res. 17, 8543-51). The appropriate sequence and length of the stem are chosen such that the hairpin structure of the probe has a melting temperature suitable for the intended annealing/extension temperature (e.g., preferably >60° C. for annealing and extension temperatures of 60° C.). However, it is preferred that little or no hairpin structure forms in the uncleaved probe, prior to binding to the target. If Tₘ of the hairpin structure is too high, e.g., much greater than the Tₘ of the target binding sequence (see preferred ranges described above), then hairpin structure can form in solution and might prevent binding to the target sequence. Tₘ of the hairpin structure can be adjusted, for example, as described below.

The hairpin forming sequence includes two self-complementary stem segments separated by a short loop segment. The stem segments are typically the same length, preferably about 4 to about 20 nucleotides, or about 4 to 15 nucleotides, and more preferably about 6 to 10 nucleotides. The loop structure can be any sequence, but preferably is not complementary to other parts of the probe or the target sequence. The loop structure is preferably from about 2 to about 10 nucleotides in length, or from about 3 to about 8 nucleotides in length, more preferably from about 4 to about 8 nucleotides in length. Thus, the overall length of the hairpin forming sequence is from about 10 nucleotides to about 50 nucleotides. The Tₘ of the hairpin structure is influenced by the length of the stem portion and number of mismatches in the stem portion. In general, each additional mismatch added to the stem region will further reduce Tₘ. Therefore, the number of mismatches can be adjusted to give a desired Tₘ. Mismatches can be positioned at any location within the stem portion of the probe, at either end or in the middle, either grouped or separated. However, the 3′ terminal base of the stem preferably is not mismatched, as this base is required for extension of the hairpin structure and signalling. Furthermore, Tₘ can be modified through the introduction of modified nucleotides, including for example minor groove binders and locked nucleic acids (LNA). Introduction of such modified nucleotides can be used to increase Tₘ or to reduce the number of mismatches required for a desired Tₘ.

The reporter binding sequence is complementary to the reporter oligonucleotide. The degree of complementarity can be full or partial, but the affinity of hybridization should be sufficiently high that the reporter oligonucleotide remains bound at the annealing/elongation temperature. Thus, similar adjustments can be made as for the hairpin stem, including varying the addition and placement of modified nucleotides or mismatches, in order to obtain the desired hybridization affinity or Tₘ. The length of the reporter binding sequence can be, for example, from about 5 nucleotides to about 40 nucleotides. The reporter oligonucleotide is preferably about the same length as the reporter binding sequence.

Labels

As used herein, the phrase “interactive pair of labels” as well as the phrase “pair of label moieties” as well as the phrase “first and second labels” refer to a pair of molecules which interact physically, optically, or otherwise in such a manner as to permit detection of their proximity by means of a detectable signal. Examples of a “pair of interactive labels” include, but are not limited to, labels suitable for use in fluorescence resonance energy transfer (FRET) (Stryer, L. Ann. Rev. Biochem. 47, 819-846, 1978), scintillation proximity assays (SPA) (Hart and Greenwald, Molecular Immunology 16:265-267, 1979; U.S. Pat. No. 4,658,649), luminescence resonance energy transfer (LRET) (Mathis, G. Clin. Chem. 41, 1391-1397, 1995), direct quenching (Tyagi et al., Nature Biotechnology 16, 49-53, 1998), chemiluminescence energy transfer (CRET) (Campbell, A. K., and Patel, A. Biochem. J. 216, 185-194, 1983), bioluminescence resonance energy transfer (BRET) (Xu, Y., Piston D. W., Johnson, Proc. Natl. Acad. Sc., 96, 151-156, 1999), or excimer formation (Lakowicz, J. R. Principles of Fluorescence Spectroscopy, Kluwer Academic/Plenum Press, New York, 1999).

The pair of labels can be either covalently or non-covalently attached to the oligonucleotide probe of the invention. Preferred are labels which are covalently attached at or near the 5′ and 3′ ends of the reporter oligonucleotide.

As used herein, references to “fluorescence” or “fluorescent groups” or “fluorophores” include luminescence and luminescent groups, respectively.

An “increase in fluorescence”, as used herein, refers to an increase in detectable fluorescence emitted by a fluorophore. An increase in fluorescence may result, for example, when the distance between a fluorophore and a quencher is increased, for example due to elimination of intraprobe hybridization, such that the quenching is reduced. There is an “increase in fluorescence” when the fluorescence emitted by the fluorophore is increased by at least 2 fold, for example 2, 2.5, 3, 4, 5, 6, 7, 8, 10 fold or more.

In certain embodiments, an increase in fluorescence or other detectable signal is driven by cleavage of the probe using a nuclease. Cleavage, for example by a 5′-flap endonuclease (e.g., FEN-1) or another nuclease or a polymerase, can be used to separate the first and second labels from each other and thus to produce the detectable signal indicating binding of the Snapback Probe to the target sequence. Alternatively, if no flap endonuclease is present in the reaction mixture, then a polymerase having 5′ to 3′ exonuclease activity, e.g., Taq polymerase, can be used for amplification.

Fluorophores

A pair of interactive labels useful for the invention can comprise a pair of FRET-compatible dyes, or a quencher-dye pair. In one embodiment, the pair comprises a fluorophore-quencher pair.

Oligonucleotide probes of the present invention permit monitoring of amplification reactions by fluorescence. They can be labeled with a fluorophore and quencher in such a manner that the fluorescence emitted by the fluorophore in intact probes is substantially quenched, whereas the fluorescence in cleaved or target hybridized oligonucleotide probes are not quenched, resulting in an increase in overall fluorescence upon probe cleavage or target hybridization. Furthermore, the generation of a fluorescent signal during real-time detection of the amplification products allows accurate quantitation of the initial number of target sequences in a sample.

In certain embodiments, the reporter oligonucleotide of the invention comprises a first labeled moiety and a second labeled moiety which serve as an interactive pair of labels comprising a fluorophore and a quencher or a FRET pair. The fluorophore or quencher can be attached to a 3′ nucleotide of the probe and the other of the fluorophore/quencher pair can be attached to a 5′ nucleotide of the probe. The interactive pair of labels may be separated, for example, by about 5, 10, 20, 30, 40, 50, 60 or more nucleotides. The fluorophore can be, for example, a FAM, R110, TAMRA, R6G, CAL Fluor Red 610, CAL Fluor Gold 540, or CAL Fluor Orange 560 and the quencher can be, for example, a DABCYL, BHQ-1, BHQ-2, or BHQ-3. In some embodiments, the detectable signal increases upon cleavage of the reporter oligonucleotide by at least 2 fold.

A wide variety of fluorophores can be used, including but not limited to: 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro -(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl) -3,6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1-sulfonic acid); DABCYL (4-((4-(dimethylamino)phenyl) azo)benzoic acid) Cy5 (Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), Quasar-670 (Bioreseach Technologies), CalOrange (Bioresearch Technologies), Rox, as well as suitable derivatives thereof.

Quenchers

As used herein, the term “quencher” refers to a chromophoric molecule or part of a compound, which is capable of reducing the emission from a fluorescent donor when attached to or in proximity to the donor. Quenching may occur by any of several mechanisms including fluorescence resonance energy transfer, photoinduced electron transfer, paramagnetic enhancement of intersystem crossing, Dexter exchange coupling, and exciton coupling such as the formation of dark complexes. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more.

The quencher can be any material that can quench at least one fluorescence emission from an excited fluorophore being used in the assay. There is a great deal of practical guidance available in the literature for selecting appropriate reporter-quencher pairs for particular probes, as exemplified by the following references: Clegg (1993, Proc. Natl. Acad. Sci., 90:2994-2998); Wu et al. (1994, Anal. Biochem., 218:1-13); Pesce et al., editors, Fluorescence Spectroscopy (1971, Marcel Dekker, New York); White et al., Fluorescence Analysis: A Practical Approach (1970, Marcel Dekker, New York); and the like. The literature also includes references providing exhaustive lists of fluorescent and chromogenic molecules and their relevant optical properties for choosing reporter-quencher pairs, e.g., Berlman, Handbook of Fluorescence Spectra of Aromatic Molecules, 2nd Edition (1971, Academic Press, New York); Griffiths, Colour and Constitution of Organic Molecules (1976, Academic Press, New York); Bishop, editor, Indicators (1972, Pergamon Press, Oxford); Haugland, Handbook of Fluorescent Probes and Research Chemicals (1992 Molecular Probes, Eugene) Pringsheim, Fluorescence and Phosphorescence (1949, Interscience Publishers, New York), all of which incorporated hereby by reference. Further, there is extensive guidance in the literature for derivatizing reporter and quencher molecules for covalent attachment via common reactive groups that can be added to an oligonucleotide, as exemplified by the following references, see, for example, Haugland (cited above); Ullman et al., U.S. Pat. No. 3,996,345; Khanna et al., U.S. Pat. No. 4,351,760, all of which hereby incorporated by reference.

A number of commercially available quenchers are known in the art, and include but are not limited to DABCYL, BHQ-1, BHQ-2, and BHQ-3. The BHQ (“Black Hole Quenchers”) quenchers are a new class of dark quenchers that prevent fluorescence until a hybridization event occurs. In addition, these new quenchers have no native fluorescence, virtually eliminating background problems seen with other quenchers. BHQ quenchers can be used to quench almost all reporter dyes and are commercially available, for example, from Biosearch Technologies, Inc (Novato, Calif.).

Attachment of Fluorophore and Quencher

In one embodiment of the invention, the fluorophore or quencher is attached to the 3′ nucleotide of the reporter oligonucleotide. In another embodiment of the invention, the fluorophore or quencher is attached to the 5′ nucleotide of the reporter. In yet another embodiment, the fluorophore or quencher is internally attached to the reporter. In some embodiments, either the fluorophore or quencher is attached to the 5′ nucleotide of the reporter and the other of said fluorophore or quencher is attached to the 3′ nucleotide of the reporter. Attachment can be made via direct coupling, or alternatively using a spacer molecule of, for example, from about 1 to about 5 atoms in length.

For the internal attachment of the fluorophore or quencher, linkage can be made using any of the means known in the art. Appropriate linking methodologies for attachment of many dyes to oligonucleotides are described in many references, e.g., Marshall, Histochemical J., 7: 299-303 (1975); Menchen et al., U.S. Pat. No. 5,188,934; Menchen et al., European Patent Application 87310256.0; and Bergot et al., International Application PCT/US90/05565. All are hereby incorporated by reference.

Each member of the fluorophore/quencher pair can be attached anywhere within the reporter oligonucleotide, preferably at a distance from the other of the pair such that sufficient amount of quenching occurs prior to cleavage of a label moiety from the reporter by the extension of the hairpin structure of the probe.

Until a label moiety is cleaved from the reporter oligonucleotide, the label moieties of the fluorophore/quencher pair are in a close, quenching relationship. For maximal quenching, the two moieties are ideally close to each other. In some embodiments, the quencher and fluorophore are positioned 30 or fewer nucleotides from each other.

Amplification of Target Nucleic Acid

In some embodiments, the probe of the invention is used to monitor or detect the presence of a target nucleic acid in a nucleic acid amplification reaction. In such embodiments, the method can be performed, for example, using typical reaction conditions for polymerase chain reaction (PCR). Two temperatures are achieved per cycle: one, a high temperature denaturation step (generally in the range of 90° C.-96° C.), lasting typically between 1 and 30 seconds, and a combined annealing/extension step (typically in the range of 50° C.-65° C., depending on the annealing temperature of the probe and primer and the polymerase chosen for the reaction), usually between 1 and 90 seconds. The reaction mixture, also referred to as the “PCR mixture”, contains a nucleic acid, a nucleic acid polymerase as described above, the oligonucleotide probe of the present invention, suitable buffer, and salts. The reaction can be performed in any thermocycler commonly used for PCR. However, preferred are cyclers with quantitative fluorescence measurement capabilities, including Taq Man 7700 AB (Applied Biosystems, Foster City, Calif.), Rotorgene 2000 (Corbett Research, Sydney, Australia), LightCycler (Roche Diagnostics Corp, Indianapolis, Ind.), iCycler (Biorad Laboratories, Hercules, Calif.) and Mx4000, Mx3000p, or Mx3005p (Stratagene, La Jolla, Calif.).

Use of a probe generally in conjunction with the amplification of a target polynucleotide, for example, by PCR, e.g., is described in many references, such as Innis et al., editors, PCR Protocols (Academic Press, New York, 1989); Sambrook et al., Molecular Cloning, Second Edition (Cold Spring Harbor Laboratory, New York, 1989), all of which are hereby incorporated herein by reference. In preferred embodiments, the binding site of the probe is located between the PCR primers used to amplify the target polynucleotide. Preferably, PCR is carried out using Taq DNA polymerase or an equivalent thermostable DNA polymerase, and the annealing temperature of the PCR is about 5° C.-10° C. below the melting temperature of the oligonucleotide probes employed.

Kits

The invention is intended to provide novel compositions and methods for amplification and/or detection as described herein. The invention herein also contemplates a kit format which comprises a package unit having one or more containers of the subject composition and in some embodiments including containers of various reagents used for polynucleotide synthesis, including synthesis in PCR. The kit may also contain one or more of the following items: polymerization enzymes (i.e., one or more nucleic acid polymerases, such as a DNA polymerase, especially a thermostable DNA polymerase), polynucleotide precursors (e.g., nucleoside triphosphates), primers, buffers, instructions, and controls. The kits may include containers of reagents mixed together in suitable proportions for performing the methods in accordance with the invention. Reagent containers preferably contain reagents in unit quantities that obviate measuring steps when performing the subject methods. One kit according to the invention also contains a DNA yield standard for the quantitation of the PCR product yields from a stained gel.

The invention also provides compositions comprising an oligonucleotide probe for detecting a target nucleic acid sequence. In some embodiments the composition comprises the probe and one or more primers for amplification of the target nucleic acid sequence. In certain embodiments the composition comprises a plurality of probes and a plurality of primers or primer pairs, which can be used, for example, to perform multiplex PCR, in which a plurality of target sequences are detected and amplified simultaneously. In other embodiments the composition comprises the probe and a nucleic acid polymerase. In still other embodiments, the composition comprises the probe, one or more primers for amplification of the target sequence, and a nucleic acid polymerase. In yet other embodiments, the composition comprises a plurality of probes, a plurality of primers or primer pairs for amplification of the target sequences detected by the plurality of probes, and a nucleic acid polymerase. In other embodiments, the composition comprises the probe and a cleavage agent, such as a nuclease. Still other embodiments of the composition include the probe, a cleavage agent, and one or more nucleic acid polymerases.

### EXAMPLES

The examples below are non-limiting and are merely representative of various aspects and features of the subject invention.

### Example 1

Use of Snapback Oligonucleotide Probe to Quantify CFTR-Specific DNA

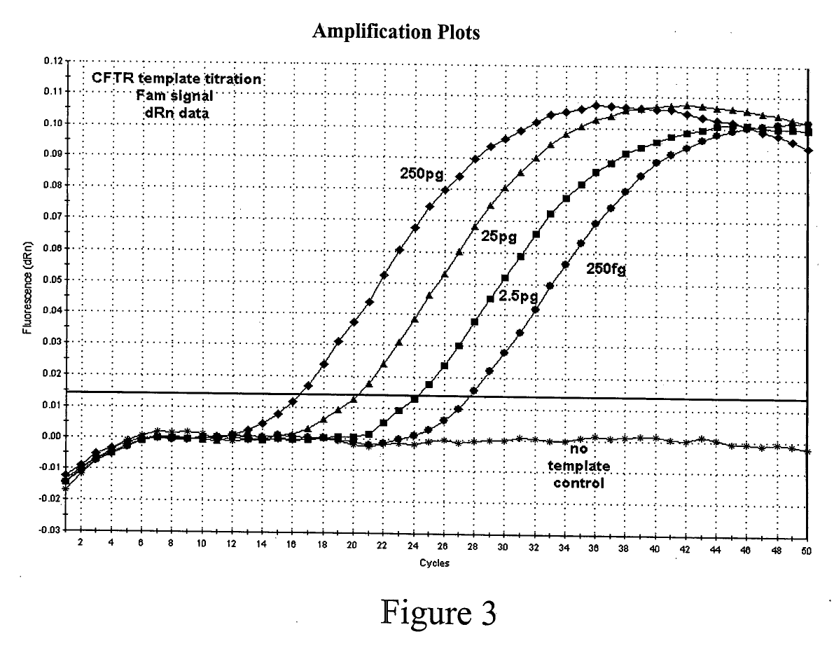

A CFTR-specific DNA template (SEQ ID NO:2, GCAGTGGGCTGTAAACTCCAGCATAGATGTGGATAGCTTGATGCGATCTGTGAGCCGAGTCTTT AAGTTCATTGACATGCCAACAGAAGGTAAACCTACCAAGTCAACCAAACC) (250 fg, 2.5 pg, 25 pg, or 250 pg) was titrated into a FullVelocity™ PCR reaction containing 400 nM CFTR-specific forward primer (SEQ ID NO:3); 200 nM CFTR-specific reverse primer (SEQ ID NO:4); 100 nM CFTR-specific Snapback probe containing a GBS (Group B Streptococcus) complementary sequence as reporter binding sequence; 100 nM GBS Reporter Oligonucleotide, labeled with 3′-FAM and quenched with 5′-BHQ-2, and 2.5 U/reaction of Full Velocity enzyme mix. The CFTR-specific Snapback probe is depicted in FIG. 2; its sequence is AGGGTTGCGATGGTTCTGTTGTAGGTACTCGTGTCTTTTGACGAGAGCTTGATGCGATCTGTGA GCCGA (SEQ ID NO:1). The CFTR-specific primers were 5′-GCAGTGGGCTGTAAACTCC-3′ (forward primer, SEQ ID NO:3) and 5′-GGTTTGGTTGACTTGGTAGG-3′, (reverse primer, SEQ ID NO:4). The experiment was conducted on an Mx3000p quantitative PCR instrument (Stratagene) with the following cycling parameters: 2 min at 95° C., followed by 50 cycles of 10 sec at 95° C., 30 sec at 60° C. Data were normalized by inclusion of 30 nM ROX reference dye in each reaction and are expressed as dRn (change in FAM fluorescence, normalized to the reference dye) with respect to cycle number. Normalized data are shown in FIG. 3. A no-template sample lacking CFTR DNA was included as a negative control. Each reaction was performed in duplicate. Full Velocity™ QPCR Master Mix is Stratagene Catalog No. 600561 and is described further in U.S. Pat. Nos. 6,528,254 and 6,548,250 (each incorporated herein by reference in its entirety).

1. An oligonucleotide probe for detecting a target nucleic acid sequence, said probe comprising a target binding sequence, a hairpin forming sequence, and a reporter binding sequence, wherein the portion of the probe comprising said hairpin forming sequence and said reporter binding sequence forms a cleavage structure when the probe is bound to a target nucleic acid, wherein said portion is cleaved off during a nucleic acid amplification reaction, wherein said hairpin forming sequence forms a hairpin structure after cleavage of said portion, and wherein extension of said hairpin structure by a nucleic acid polymerase results in release of a label moiety from said reporter, resulting in a detectable signal.

2. The oligonucleotide probe of claim 1, wherein the 3′ end of said reporter binding sequence is covalently linked to the 5′ end of said hairpin forming sequence, and wherein the 3′ end of said hairpin forming sequence is covalently linked to the 5′ end of said target binding sequence.

3. The oligonucleotide probe of claim 2, further comprising a linker sequence between said reporter binding sequence and said hairpin forming sequence.

4. The oligonucleotide probe of claim 3, wherein the linker is about 3 nucleotides in length.

5. The oligonucleotide probe of claim 1, wherein the hairpin forming sequence comprises a first stem sequence, a loop sequence, and a second stem sequence.

6. The oligonucleotide probe of claim 1, wherein the target binding sequence is from about 5 to about 60 nucleotides in length.

7. The oligonucleotide probe of claim 1, wherein the hairpin forming sequence is from about 10 to about 50 nucleotides in length.

8. The oligonucleotide probe of claim 1, wherein the reporter binding sequence is from about 5 to about 40 nucleotides in length.

9. The oligonucleotide probe of claim 5, wherein the first stem sequence is from about 4 to about 20 nucleotides in length.

10. The oligonucleotide probe of claim 5, wherein the second stem sequence is from about 4 to about 20 nucleotides in length.

11. The oligonucleotide probe of claim 5, wherein the loop sequence is from about 2 to about 10 nucleotides in length.

12. The oligonucleotide probe of claim 1 which is from about 30 to about 130 nucleotides in length.

13. The oligonucleotide probe of claim 1, wherein said cleavage structure is a 5′ flap.

14. A double-stranded probe comprising the oligonucleotide probe of claim 1 and a reporter oligonucleotide that binds to the reporter binding sequence of the oligonucleotide probe of claim 1.

15. The reporter oligonucleotide of claim 14 comprising a pair of label moieties.

16. The reporter oligonucleotide of claim 15, wherein one label moiety of said pair is a quencher and the other label moiety of said pair is a fluorophore, wherein said fluorophore is quenched when the reporter oligonucleotide is bound to the reporter binding region of said oligonucleotide probe, and wherein said fluorophore is unquenched upon extension of said hairpin structure by a nucleic acid polymerase.

17. The reporter oligonucleotide of claim 16 that forms a cleavage structure upon binding to said reporter binding sequence.

18. The reporter oligonucleotide of claim 17, wherein said cleavage structure is a flap.

19. The reporter oligonucleotide of claim 18, wherein said flap comprises one of said label moieties, and wherein extension of said hairpin structure by a nucleic acid polymerase results in cleavage of said flap.

20. A method of detecting a nucleic acid amplification product, comprising
amplifying a target nucleic acid sequence in the presence of the double-stranded probe of claim 12 using (a) a pair of primers specific for the target nucleic acid sequence and (b) a nucleic acid polymerase having a 5′ to 3′ exonuclease activity; and
detecting the signal produced by the cleavage of a label moiety of said probe, wherein the signal indicates detection of an amplification product derived from said target sequence.

21. The method of claim 20, wherein the polymerase is a Taq polymerase.

22. A method of detecting a nucleic acid amplification product, comprising
amplifying a target nucleic acid sequence in the presence of the double-stranded probe of claim 14 using (a) a pair of primers specific for the target nucleic acid sequence, (b) a nucleic acid polymerase, and (c) a flap endonuclease; and
detecting the signal produced by the cleavage of a label moiety of said probe, wherein the signal indicates detection of an amplification product derived from said target sequence.

23. The method of claim 22, wherein the polymerase substantially lacks 5′ to 3′ exonuclease activity.

24. The method of claim 22, wherein the flap endonuclease is FEN-1.

25. The method of claim 20, wherein the step of amplifying is performed using a polymerase chain reaction.

26. The method of claim 22, wherein the step of amplifying is performed using a polymerase chain reaction.

27. A kit for detecting a product of a nucleic acid amplification, comprising the oligonucleotide probe of claim 1, packaging, and instructions for use.

28. The kit of claim 27 further comprising the reporter oligonucleotide of claim 14.

29. The kit of claim 27 further comprising a pair of primers specific for amplification of the target nucleic acid sequence.

30. The kit of claim 29 further comprising a nucleic acid polymerase substantially lacking 5′ to 3′ exonuclease activity and a flap endonuclease.

31. The kit of claim 29 further comprising a nucleic acid polymerase having 5′ to 3′ exonuclease activity.
